(19) United States
(12) Patent Application Publication
(10) Pub. No.: US 2007/0038123 A1
(43) Pub. Date: Feb. 15, 2007

(54) Optical probe for Raman scattering from arterial tissue

(75) Inventor: Stephen Fulghum, Marblehead, MA (US)

Correspondence Address: WEINGARTEN, SCHURGIN, GAGNEBIN & LEBOVICI LLP, TEN POST OFFICE SQUARE, BOSTON, MA, 02109, US

(73) Assignee: Newton Laboratories, Inc.

(21) Appl. No.: 11/444,980

(22) Filed: Jun. 1, 2006

Related U.S. Application Data
(60) Provisional application No. 60/686,601, filed on Jun. 2, 2005.

(51) Int. Cl. A61B 6/00

(52) U.S. Cl. 600/476; 600/473

(57) ABSTRACT

The present invention related to a Raman artery probe diagnostic system and a method of using the system for the diagnosis of vascular disease. A side looking catheter having light delivery and collection optical fibers can deliver infrared light in a radial direction from the catheter to diagnose atherosclerotic plaques in the coronary arteries. A guidewire or guide catheter can be used to deliver the catheter to the region of interest within the artery to evaluate vulnerable plaques.

### CROSS REFERENCE TO RELATED APPLICATIONS

This application claims the priority of U.S. Provisional Application No. 60/686,601 filed Jun. 2, 2005 entitled, OPTICAL PROBE FOR RAMAN SCATTERING FROM ARTERIAL TISSUE, the whole of which is hereby incorporated by reference herein.

### BACKGROUND OF THE INVENTION

Optical methods have been developed for the measurement of tissue. Raman spectroscopy, due to its ability to discern the chemical components of tissue has been developed to aid in the diagnosis of disease. Optical fiber probes have been used in conjunction with this method to deliver and collect light in regions of the human body to provide in vivo diagnostic procedures. Small diameter catheters have been proposed for use within the coronary arteries in order to diagnose atherosclerotic lesions.

Spectrometers and detectors are used to acquire spectral data that is processed to provide diagnostic information. However, due to the weakness of the Raman signals returning from the tissue, it remains difficult to collect enough light such that a reliable diagnostic procedure can be deployed in a clinical setting. Thus, a continuing need exists for improvements in methods and systems for the use of optical probes for diagnostic applications.

### BRIEF SUMMARY OF THE INVENTION

The present invention relates to a side-looking optical probe or catheter to detect Raman scattering from artery walls. A preferred embodiment of the probe utilizes an axially symmetric design with a lumen centered on the axis so that the probe can be inserted over a guidewire which has been previously placed into the artery. Such guidewires can be used to control delivery of the catheter during insertion into a patient. In a preferred embodiment four optical fibers, equally spaced around the central lumen, transmit excitation light to the distal tip. A reflective optical element such as sapphire axicon at the distal tip of the probe, for example, directs this excitation light sideways to the tissue. Within each quadrant, a plurality of optical fibers receives scattered Raman light from the tissue and transmits it to the proximal end of the probe for analysis. The excitation fibers and collection fibers are placed at separate radial distances from the central axis. This spacing allows filters to be coated onto the base of the axicon to pass only excitation light to the tissue (blocking excitation fiber fluorescence) and to pass only Raman scattering to the collection fibers (blocking excitation light).

The convex reflective surface of the axicon disperses the excitation light azimuthally but not axially, providing near complete coverage of the inner wall of the artery. The central lumen has sufficient diameter to allow for the passage of a balloon catheter and/or guidewire for occluding the artery directly in front of the distal tip. A saline flush through the central lumen, around the balloon guidewire, clears blood from the probe to wall region. A full scan of the artery wall is provided by collecting Raman-scattered light from all four quadrants as the probe is withdrawn through the arterial segment of interest. The probe thus does not need to be rotated or need rotating elements within the probe to accomplish a full scan.

The embodiments of the present invention can be used in the diagnosis and treatment of vulnerable plaques and in particular the location of a fibrous cap and the underlying lipid pool that are characteristics of such plaques.

The following figures show the construction methods and physical characteristics of the probe.

### BRIEF DESCRIPTION OF THE DRAWINGS

FIGS. 1A-1F illustrate cross sectional views of an optical probe in accordance with a preferred embodiment of the invention.

FIGS. 2A-2C illustrate detailed cross sectional views illustrating a method of forming a filtered side looking optical element for an optical probe in accordance with a preferred embodiment of the invention.

FIG. 3 illustrates a preferred embodiment of a Raman artery probe system in accordance with the invention.

FIG. 4 illustrates a diagnostic procedure in accordance with the invention.

### DETAILED DESCRIPTION OF THE INVENTION

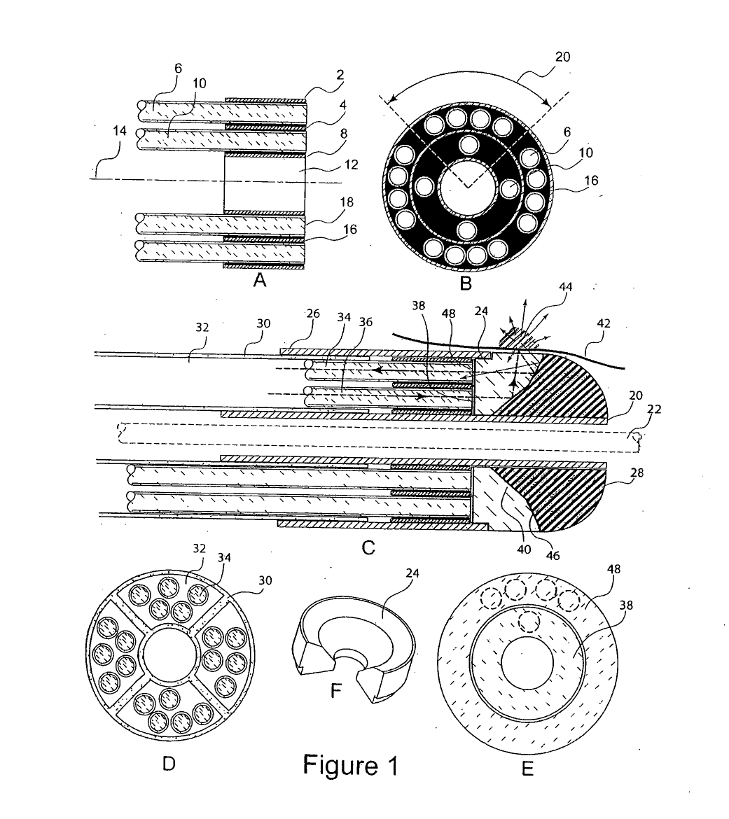

FIG. 1A shows a ferrule including three concentric thin-wall tubes of drawn stainless steel which hold the optical fibers in their correct radial and azimuthal positions. The outer tube, 2, and middle tube, 4, constrain the receiver fibers, 6, to the outer radius closest to the artery wall. The middle tube, 4, and central tube, 8, constrain the excitation fibers, 10, to the inner radius. The lumen of the central tube, 8, is centered on the axis of the probe, 14, and is kept free for the passage of a guide wire. The fibers, 6 and 10, and the ferrule tubes, 2, 4 and 8, are epoxied together, 16, into a unit so that the ends of the fibers can be optically polished together to form a single plane, 18.

FIG. 1B shows an end view of the completed ferrule indicating how the excitation fibers, 10, and receiver fibers, 6, are arranged within a given quadrant, 20. The Raman excitation source is a laser, so that only a single fiber, 10, is necessary to deliver excitation light to a given quadrant. The Raman-scattered light from the tissue, however, radiates into. 4 n steradians so that multiple receiver fibers, 6, are necessary to collect as much of this scattered light as possible.

FIG. 1C shows the assembly of the polished optical fiber bundle, 1A, with the rest of the optical and mechanical components at the distal tip of the probe. The polished fiber bundle is slipped over a thin-wall, stainless steel, core tube, 20, which serves to align the tip components and provide the shear strength for the assembly. The center of the core tube, 20, remains open for the guide wire, 22, to pass through when the probe is in use. The next component to be assembled is the sapphire axicon, 24, which is also slipped over the core tube and epoxied to the flat face of the optical fiber bundle, 1A, with a clear optical epoxy such as EPO-TEK 301. At the same time, the outer thin-wall, stainless steel tube, 26, is epoxied to the outer perimeter of the optical fiber bundle, 1A, and a notch ground into the edge of the sapphire axicon, 24. This outer tube, 26, serves to prevent fluids from reaching the internal spaces of the probe fiber bundle. The rounded probe tip, 28, is an epoxy bead designed to simultaneously seal against the reflective back wall of the sapphire axicon, 24, and the core tube, 20, for strength, protection of the axicon, fluid sealing and providing a smooth surface to ease insertion of the probe into an artery. The tip, 28, can also be made of a solid material and bonded in place. A multi-lumen, flexible, fiber sheath, 30, such as those made by Zeus, Inc., carries the optical fibers within the lumens, sealed from body fluids, with sufficient room to allow them some relative motion for flexibility. The four separate lumens, 32, shown in FIG. 1D, also keep the quadrant fibers separated, preventing fibers from crossing which can lead to fiber breakage. The fiber sheath is bonded over the core tube, 20, on its internal surface and to the outer tube, 26, on its outside surface to complete the fluid seal separating its hollow lumens from body fluids.

Systems and method for using the probe of the present invention are described in greater detail in U.S. Pat. Nos. 6,690,966 and 6,697,665 as well as in U.S. application Ser. No. 10/407,923 filed on Apr. 4, 2003, the entire contents of the above patents and application being incorporated herein by reference. A preferred embodiment of the present invention uses a ball lens to provide a forward looking probe with a single central delivery fiber and a peripheral ring of collection fibers. A portion of the lens surface can be removed to provide a cylindrical outer surface that can be bonded or attached to an inner wall of a probe body. This device can be inserted within an endoscope channel for diagnostic procedures in the lung, colon or GI tract in conjunction with standard or fluorescence imaging.

FIG. 1C also shows the optical path for the Raman excitation light on its way to the tissue and for the Raman scattered light from the tissue on its way back to the analyzer as indicated by the heavy dotted line in FIG. 10. It should be noted that the dotted line is only one of many such paths that the light can take within the 8.7° internal acceptance angle of a typical fused silica optical fiber shown in this embodiment.

Note that a further embodiment of the invention uses a guide catheter instead of a guidewire to deliver the catheter to the location of interest. In this embodiment a single curved distal surface encloses the end of the catheter body. Alternatively the catheter can employ a mechanical or electromechanical actuator to steer the catheter into position.

The excitation light enters the probe tip through the excitation fiber, 36. The excitation light passes through a thin-film filter, 38, which is coated on the back surface of the sapphire axicon, 24, in the form of an annular ring as shown in FIG. 1E. This thin-film filter, 38, has a bandpass centered on the Raman excitation laser wavelength, generally in the infrared or near infrared range of 700 nm to 1000 nm, and serves to block Raman-scattered light from the excitation fiber (at longer wavelengths) from being collected in addition to the Raman scattering from the tissue. After the bandpass filter, 38, the excitation light reflects off of an annular facet, 40, on the front surface of the sapphire axicon, 24. This facet is angled to direct the excitation light to the tissue, 42, at a point on the tissue, 44. This facet can either be flat, as drawn, or elliptical to better concentrate the excitation light on the tissue at point 44. The nominal angle of the facet, 40, will not support total internal reflection so this facet must be coated on the front face of the axicon with a metallic layer such as aluminum or preferably gold for the longer wavelengths typically used for Raman excitation. This metallic layer also allows the front surface of the axicon to be bonded directly to the tip, eliminating air layers which are difficult to keep clean and which present a potential entrance point for the leakage of body fluids.

The excitation light exits the distal tip of the probe through the annular window provided by the sapphire axicon and strikes the tissue at position 44. The excitation light diffuses into the tissue for some distance, with a small amount of Raman scattering occurring at all points along the light path. The Raman scattering radiates into 4 n steradians and thus provides a diffuse source, indicated by the small arrows radiating in all directions from a volume around position 44.

A fraction of the Raman scattered light is reflected off of the outer front facet, 46, of the sapphire axicon, 24 shown in perspective in FIG. 1F, into the conical collection angles of the receiver fibers, 34. Before entering the receiver fiber, 34, the light from the tissue must pass through the thin-film filter, 48, coated onto the back side of the sapphire axicon, 24, as shown in FIG. 1E. This filter, 48, passes the longer wavelengths of the Raman scattered light but reflects most of the narrowband excitation light which has scattered off of the tissue without changing wavelengths. By blocking this excitation wavelength the additional Raman signal due to the long return path of fused silica will be avoided. Both the bandpass filter, 38, and the longpass filter, 48, are necessary to collect a Raman spectrum of the tissue with a high signal-to-noise ratio.

The specific angles and shapes of the facets, 40 and 46, of the sapphire axicon can be optimized to obtain the highest return signal into the receiver fibers. At least two facets are required because the excitation fiber and the receiver fiber are at different radii from the axis of the probe and must be directed at a common point on the tissue. This is different from the case of a fluorescence probe where both the excitation fibers and receiver fibers, which do not need to be filtered, can be placed at the same radius. For a fluorescence probe the optimum surface for the axicon is a single toroidal ellipse. Further details regarding this embodiment can be found in U.S. Application No. 60/686,600 filed Jun. 2, 2005, by Fulghum, et al., the entire contents of which is incorporated herein by reference.

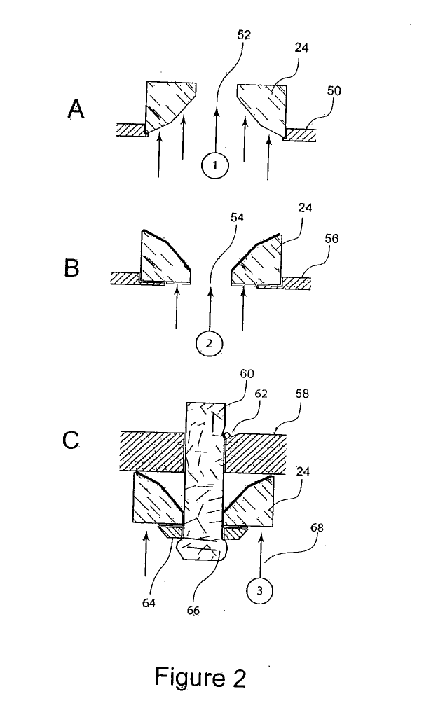

Placing the bandpass filter, 38, and the longpass filter, 48, onto the back surface of the axicon, 24, is a major simplification in the design of this Raman probe compared to previous, forward-directed designs in which these filters were placed on separate substrates. These axicons are small, however, being on the order of two millimeters in diameter. To coat them for this use requires three separate steps as shown in FIGS. 2A, 2B and 2C. In the first step, 2A, the axicon, 24, is placed into a fixture, 50, within a vacuum deposition chamber to be coated with a metallic layer of aluminum or preferably, gold, 52, on its front face that is about 1-10 microns thick. The bandpass filter, 38, is coated, 54, onto the back surface of the axicon, 24, by placing it into a second mask, 56, which prevents the deposition stream, 54, from reaching the outer edge of the back surface where the longpass filter, 48, will be deposited. For the third and final coating of the axicon, 24, the substrate is held in a fixture, 58, into which a soft metallic wire, 60, is placed by staking, 62. This wire, 60, is a close fit to the inside bore of the axicon, 24, so that a precision washer, 64, can be held in place by crimping, 66, the end of the wire with all components in place. The precision washer serves as a mask to prevent the longpass thin film filter coating deposition stream, 68, from overlaying the bandpass coating. A large number of the axicons can be coated within each run in this fashion. Such thin film dielectric filters are provided by Semrock, Rochester, N.Y.

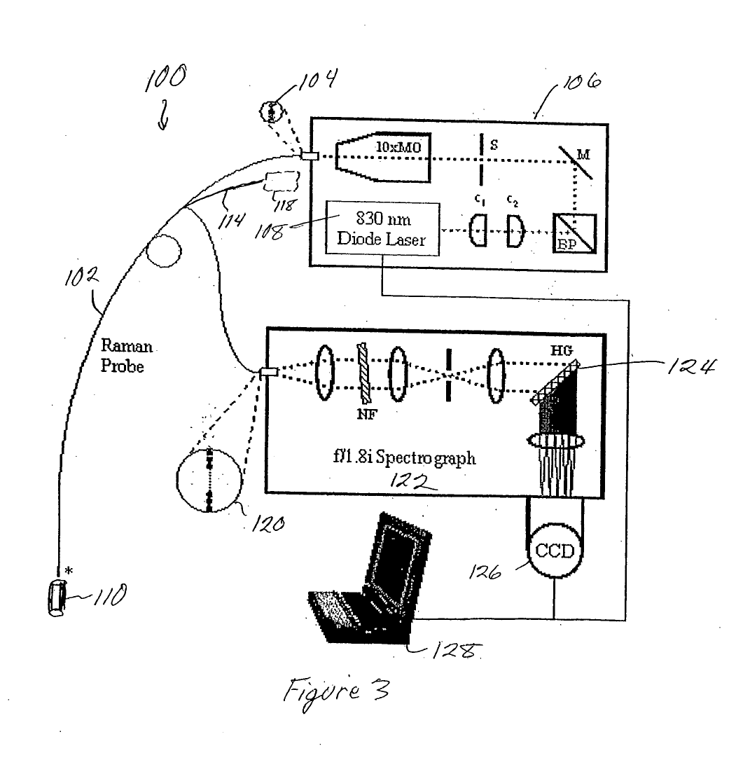

A schematic of the clinical system 100 is shown in FIG. 3. Light from an 830 nm diode laser 108 (Process Instruments, Salt Lake City, Utah, or Sacher Lasertechnik, Germany) is collimated by two cylindrical lenses (c1, c2), directed through a bandpass filter (BP, Kaiser), redirected by a gold coated mirror (M) and focused onto the Raman probe excitation fibers 104 by a 10× microscope objective (Newport, Irvine, CA). Other light sources emitting wavelengths in a range of 700-1000 nm and preferably between 800 nm and 900 nm, can also be used. The proximal linear array of collection fibers 120 from the Raman probe 102 are input to the f/1.8 spectograph 122 which collimates the light before it is notch filtered (NF), focused onto a slit and re-collimated for dispersion by the holographic grating 124 (HG). Finally, the dispersed light is focused onto a liquid nitrogen cooled, back-illuminated, deep depletion CCD detector 126, which is interfaced with a laptop computer or other computer 128 for processing and display. A slit (S) allows the laser excitation out of the probe during data acquisition. This reduces laser exposure to the patient and increases safety for the users. Further, the laser interfaces with the computer for more accurate control of the laser power, monitoring of laser power and incorporation of a feedback loop automatically sets the power output from the probe to the set level independent of the system optics alignment.

The system provides a stand-alone clinical instrument, controlled by a dedicated computer without adjustments from a front panel. The Sacher Lasertechnik diode laser operates at 830 nm and is temperature-stabilized. The linewidth of this laser system is far below the nominal 1 cm⁻¹ (30 GHz) resolution required for interpreting the Raman spectra. Typical drift in 20 seconds is only 10 MHz with a 24 hour drift of less than 1 GHz. The system does not need periodic wavelength adjustments of either the laser or notch filters. The Sacher Lasertechnik Pilot PC500 OEM power supply is microprocessor-operated and designed for rough environments with slow start and interlock protections built in. The Kaiser Holospec f/1.8I is an imaging holographic grating spectrometer with an integrated SuperNotch-Plus input filter to reduce 830 nm excitation laser leakage by more than six orders or magnitude. The Apogee AP47p cooled-CCD camera utilizes a Marconi CCD47-10 back-thinned, 13.3 mm×13.3 mm CCD with a 23% quantum efficiency at 975 nm (1800 cm⁻¹ from 830 nm). These components are provided in an enclosure suitable for a clinical environment and an embedded computer for system control, data storage and data analysis. The catheter can optionally be positioned with a guide catheter 110, or guidewire and used with saline delivery tube 114 and fluid source 118.

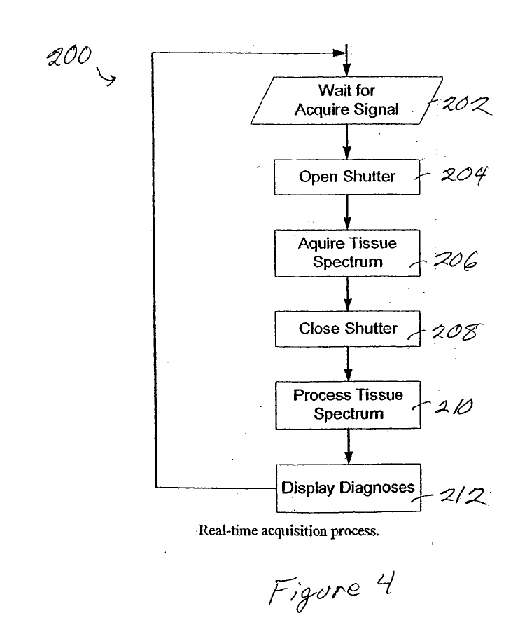

FIG. 4 illustrates the flow 200 of the methods used in acquiring data for in vivo Raman spectral diagnosis in accordance with a preferred embodiment of the present invention. Prior to the clinical procedure, calibration spectra are acquired to characterize the system performance. The spectrum of a Teflon standard is obtained to determine the expected signal with the desired excitation power. During the procedure, spectra of an identical sterilize Teflon block are taken with the sterilized Raman probe within a feedback algorithm. Automated adjustments to the laser power continue until the target Teflon intensity is obtained, or until a pre-determined threshold power is reached. Once the correct power is set, acquisition of tissue spectra is enabled. The laser is blocked by a shutter until data accumulation is initiated. The start 202 of an acquisition opens the shutter per step 204, collects the spectrum per step 206 and closes the shutter per step 208. Collected data is then processed 210 and displayed 212 in real-time along with the spectral diagnosis. The system is then ready to examine the next tissue site.

The claims should not be read as limited to the described order or elements unless stated to the effect. Therefore all embodiments that come within the scope and spirit of the following claims and equivalents thereto are claims as the invention.

What is claimed is:

1. An optical probe for arterial diagnosis with Raman scattering comprising:
a fiber optic catheter having a first plurality of delivery fibers and a second plurality of collection fibers; and
a reflective optical element at a distal end of the catheter that reflects light from the delivery fibers in a radial direction with a first reflecting surface positioned at a first angle and that reflects light into the collection fibers with a second reflecting surface at a second angle.

2. The optical probe of claim 1 further comprising a guidewire lumen.

3. The optical probe of claim 1 further comprising a laser light source coupled to a proximal end of the delivery fibers having a wavelength of 700 nm or longer.

4. The optical probe of claim 1 further comprising a spectrometer and a detector.

5. The optical probe of claim 1 wherein the reflective optical element comprises an axicon having a dielectric thin film filter.

6. The optical probe of claim 1 wherein the delivery fibers are positioned at an inner radius and the collection fibers are positioned at an outer radius within a tubular catheter body.

7. The optical probe of claim 1 wherein the catheter has a distal retainer that holds a distal end of the optical fiber in a fixed orientation and a tubular flexible catheter body having a plurality of lumens extending longitudinally within the catheter body, each lumen having a plurality of optical fibers.

8. The optical probe of claim 1 further comprising a computer connected to a detector, the computer processing spectral data to diagnose tissue.

9. An optical probe for tissue diagnosis comprising:
a probe; and
a reflective optical element at a distal end of the probe that reflects light from an axial direction in the probe in a radial direction with a first reflecting surface positioned at a first angle and with a second reflecting surface at a second angle.

10. The optical probe of claim 9 further comprising a guidewire lumen.

11. The optical probe of claim 9 further comprising a laser light source coupled to a proximal end of delivery fibers within the probe having a wavelength of 700 nm or longer.

12. The optical probe of claim 9 further comprising a spectrometer and a detector.

13. The optical probe of claim 9 wherein the reflective optical element comprises an axicon having a dielectric thin film filter.

14. The optical probe of claim 9 further comprising delivery optical fibers and collection fibers within a tubular probe body.

15. The optical probe of claim 14 wherein the probe has a distal retainer that holds a distal end of the optical fiber in a fixed orientation and a tubular flexible probe body having a plurality of lumens extending longitudinally within the probe body, each lumen having a plurality of optical fibers.

16. The optical probe of claim 9 further comprising a computer connected to a detector, the computer processing spectral data to diagnose tissue.

17. A method for using an optical probe for diagnosis comprising:
providing a fiber optic probe having a first plurality of delivery fibers and a second plurality of collection fibers; and
reflecting light with a reflective optical element at a distal end of the probe that reflects light from the delivery fibers in a radial direction with a first reflecting surface positioned at a first angle and with a second reflecting surface at a second angle.

18. The method of claim 17 further comprising providing a reflective optical element including an axicon having a dielectric thin film filter.

19. The method of claim 17 further comprising providing a distal retainer that holds a distal end of the optical fiber in a fixed orientation and a tubular flexible catheter body having a plurality of lumens extending longitudinally within the catheter body, each lumen having a plurality of optical fibers.

20. The method of claim 17 further comprising processing spectral data with a computer connected to a detector to diagnose tissue.
